(19) United States
(12) Patent Application Publication
(10) Pub. No.: US 2026/0240660 A1
(43) Pub. Date: Aug. 20, 2026

(54) MECHANICAL IMPLANT FOR HIP AND SHOULDER ARTHROPLASTY

(71) Applicant: Georgios CHRISTOFORIDIS, VIRONAS ATTIKIS (GR)

(72) Inventor: Georgios CHRISTOFORIDIS, VIRONAS ATTIKIS (GR)

(21) Appl. No.: 19/471,041

(22) PCT Filed: Aug. 2, 2023
(86) PCT No.: PCT/GR2023/000040

§ 371 Date: Sep. 30, 2025

(30) Foreign Application Priority Data
Apr. 21, 2023 (GR) 20230100336

(51) Int. Cl. A61F 2/40; A61F 2/30; A61F 2/36

(52) U.S. Cl. CPC A61F 2/4014; A61F 2/30767; A61F 2/3609; A61F 2/3662; A61F 2/4059; A61F 2002/30179; A61F 2002/30331; A61F 2002/30383; A61F 2002/30433; A61F 2002/305; A61F 2002/30555; A61F 2002/3093; A61F 2002/3611; A61F 2002/3627; A61F 2002/3652; A61F 2002/4029; A61F 2002/4044; A61F 2310/00023; A61F 2310/00796

(57) ABSTRACT

The invention relates to a mechanical implant for hip or shoulder arthroplasty. A mechanical implant consisting of a neck (1) and an end (2), which is inserted into the lumen of the patient's bone, ends in a movable mechanical part (3), which is adapted to the metaphysis of the femur or humerus, and two movable parts (4), which are used to adapt the end (2) onto the neck (1). The two parts, the neck (1) and the movable mechanical part (3) are joined together with screws (5). The invention offers increased stability and allows easier replacement of the neck section (1) in cases of revision.

### FIELD OF ART

The invention relates to the technical field of surgical tools, devices or methods and more specifically to artificial substitutes or replacements for parts of the body and even more specifically to implants for the hip joint at the place of the femoral head and for the shoulder joint at the place of the humeral head.

### BACKGROUND OF THE INVENTION

The mechanical implant for partial or total hip and shoulder arthroplasty disclosed in the present invention has not been disclosed in the prior art.

Under normal conditions the femoral head of a healthy person articulates with the acetabulum of the pelvis, allowing a wide range of motion of the hip in multiple planes. Many diseases, such as for example osteoarthritis, can lead to a disorder of the joint's function, due to the alteration of the joint cartilage and consequent pain, deformation and loss of its functionality. Total hip arthroplasty involves the total replacement of both the femoral head and neck of the bone, as well as the acetabulum of the pelvis, usually with metal and polyethylene alloy implants. This orthopedic operation is performed to reduce the severe pain, which comes from the friction of the degenerated cartilage, with the aim of restoring functionality and improving the quality of life.

So far, in the case that a patient suffering from chronic joint pain due to arthritis chose to undergo joint replacement surgery, the orthopedic surgeon would replace the existing joint surfaces with artificial implants. These implants must adhere to the patient's natural bone. The methods by which the implant adheres to the bone are largely divided into two main categories, the methods using cement and the methods that do not require the use of cement. The use of cement during implant placement requires the addition of a layer of bone cement, usually an acrylic polymer called polymethyl methacrylate (PMMA), between the patient's natural bone and the prosthetic joint component.

The downside to using bone cement is that it can degrade over time and pieces of the cement can break off, potentially causing problems. Breakdown of the cement can cause the artificial joint to loosen, which may cause the need for another joint replacement surgery (revision surgery). Residual cement can irritate the surrounding soft tissue and cause inflammation. Although rare, cement can enter the bloodstream and end up in the lungs, a condition that can be life-threatening. In addition, the application of bone cement in closed bone cavities, such as the femur, is associated with the development of cement application syndrome, which is characterized by sudden hypotension, hypoxia, and even cardiac arrest, due to embolism of fat globules and marrow elements in the lungs. Finally, necrosis and fever are frequent phenomena for patients undergoing related operations with the use of cement.

On the other hand, cementless implant placement methods, also called press-fit methods, require implants that have a rough surface or porous coating that encourages natural bone to grow over it. The new bone growth will only extend 1 or 2 mm, so the surgeon must use special tools to shape the natural bone to fit the implant comfortably.

Some implants have screws or plates that help hold the bone and prostheses in place until new bone growth can create a more secure attachment. The disadvantages of these methods, which do not use cement, are that they require healthy bone. Patients with low bone density, due to osteoporosis for example, may not be suitable for these methods. An additional disadvantage is that it can take up to three months for bone material to grow around the new implant. Because it takes time for the natural bone to fully attach to the implant, experts debate whether or not patients should put off allowing all of their weight to be put onto new joints. A further disadvantage that may occur frequently is due to the shocks for applying the implant, which may even cause the bone to break. Additionally, over time, the end of the implant may sink into the bone and the implant may lose its functionality. Another common undesired complication involves the opening of the bone lumen, causing the implant to loosen and gain unwanted mobility within the lumen. Finally, collared femoral prostheses may prevent full impaction of the femoral prosthesis, causing satisfactory vertical stability but rotational instability of the prosthesis.

It is thus an object of the present invention to advantageously address the aforementioned disadvantages and shortcomings of the prior art by proposing a mechanical implant for partial or total hip and shoulder arthroplasty.

It is a further object of the present invention to provide a mechanical implant for partial or total hip and shoulder arthroplasty that conforms to the lumen of the bone and provides stability, both during application and during the life of the implant.

Another object of the invention is to provide a mechanical implant for partial or total hip and shoulder arthroplasty, which has a movable mechanical part, which adapts to the metaphysis of the femur or humerus.

It is a further object of the present invention to provide a mechanical implant for partial or total hip and shoulder arthroplasty which ensures that over time the end of the implant will not sink into the bone lumen.

A further object of the invention is to provide a mechanical implant for partial or total hip and shoulder arthroplasty, which in case of revision allows for easier replacement of the entire segment.

An additional advantage of the mechanical implant of the invention is the fact that it is fully adjustable, allowing it to be adapted to any known type of bone, ensuring an excellent fit.

These and other objects, features and advantages of the invention will become apparent in the following detailed description.

### BRIEF DESCRIPTION OF THE DRAWINGS

The invention will become apparent to those skilled in the art by reference to the accompanying drawings, in which it is illustrated in an exemplary, non-limiting manner.

FIG. 1 shows in an expanded view the mechanical implant of the invention and the parts that make it up.

FIG. 2 shows an illustrative embodiment of the mechanical implant for partial or total hip and shoulder arthroplasty.

FIG. 3 (a)-(b) show a plan view and a side view of the mechanical part with the end inserted into the bone lumen.

FIG. 4 shows the neck forming part of the mechanical implant of the invention.

### DETAILED DESCRIPTION OF A PREFERRED EMBODIMENT

Referring now to the accompanying drawings, we will describe exemplary embodiments of the mechanical implant used for partial or total hip and shoulder arthroplasty.

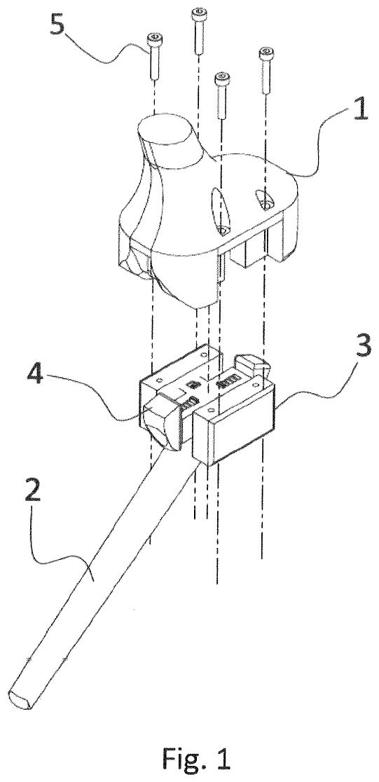

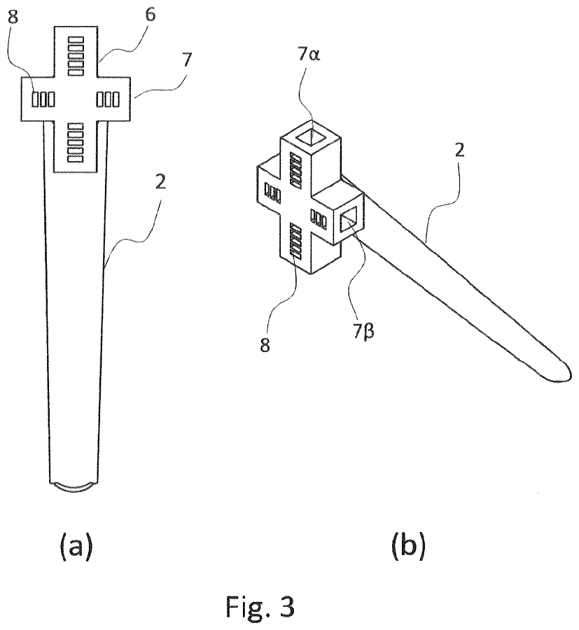

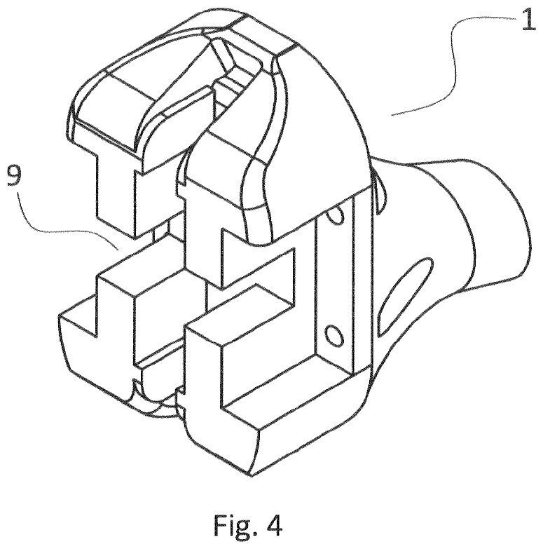

The mechanical implant of the invention consists of two sub-parts, the neck (1), FIG. 4 and the end (2), FIG. 3(a), which is inserted into the lumen of a patient's bone. The end (2) terminates in a movable mechanical part (3), which is adapted to the metaphysis of the femur or humerus, and two movable parts (4), which are used to fit the end (2) onto the neck (1). The two parts, the neck (1) and the movable mechanical part (3) are joined together with screws (5), FIG. 1.

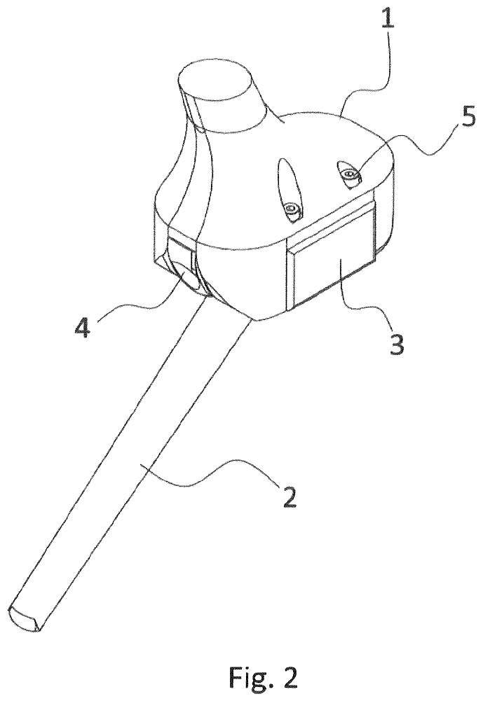

The end (2) has a cross-shaped structure (6) with four holes (7), the movable mechanical part (3) applied in the vertical holes (7a), FIG. 3(b), in relation to the end (2), while in the horizontal holes (7b) in relation to the end (2) the two movable parts (4) apply. The cruciform structure (6) has, on each side, slits (8) in which the mechanical part (3) and the movable parts (4) fit and are adjusted, respectively, in the metaphysis of the bone. The neck (1) in its lower part, has a configuration with notches (9), which fit the cruciform structure (6) for the neck (1) to integrate in the end (2) and respectively in the movable mechanical part (3) with the movable parts (4) in order to create the mechanical implant, FIG. 2.

The end (2) as well as the neck (1) is advantageously, but not limitingly, made of titanium, while the movable mechanical part (3) having a coating of hydroxyapatite.

It should be noted at this point that the description of the invention was made with reference to illustrative embodiments, to which it is not limited. Thus, any change or modification in terms of shape, dimensions, morphology, materials used and construction and assembly components, as long as they do not constitute a new inventive step and do not contribute to the technical development of what is already known, are considered included in the aims and objectives of the present invention.

1. A mechanical implant for hip and shoulder arthroplasty, consisting of a neck (1) and an end (2), which enters the lumen of a patient's bone, terminates in a movable mechanical part (3), which adapts to the metaphysis of the bone and with two movable parts (4), where the end (2) has a cross-shaped structure (6) with four holes (7), where the movable mechanical part (3) applies in the vertical holes (7a) in relation to the end (2) and in the horizontal holes (7b) in relation to the end (2) apply the two movable parts (4), characterized in that the cruciform structure (6) has on each side of it, slits (8) in which the movable mechanical part (3) and the two movable parts (4) are applied and are adjusted accordingly according to the metaphysis of the bone, the neck (1) in its lower part having a configuration with notches (9), which fit the cross-shaped structure (6) for the neck (1) to integrate into the end (2) and the movable mechanical part (3) with the two movable parts (4).

2. A mechanical implant for hip and shoulder arthroplasty according to claim 1, characterized in that the end (1) and the neck (2) are made of titanium.

3. A mechanical implant for hip and shoulder arthroplasty, according to claim 1, characterized in that the mechanical part (3) has a coating of hydroxyapatite.
